(19) United States
(12) Patent Application Publication
(10) Pub. No.: US 2021/0355948 A1
(43) Pub. Date: Nov. 18, 2021

(54) CEILING FAN WITH AIR CLEANER

(71) Applicant: DELTA T, LLC, Lexington, KY (US)

(72) Inventor: Stephen Alexander Harper, Lexington, KY (US)

(21) Appl. No.: 17/321,712

(22) Filed: May 17, 2021

Related U.S. Application Data
(60) Provisional application No. 63/025,621, filed on May 15, 2020.
(60) Provisional application No. 63/029,105, filed on May 22, 2020.
(60) Provisional application No. 63/038,446, filed on Jun. 12, 2020.
(60) Provisional application No. 63/045,882, filed on Jun. 30, 2020.
(60) Provisional application No. 63/060,826, filed on Aug. 4, 2020.

(51) Int. Cl. F04D 25/08; F04D 29/70; F24F 8/22

(52) U.S. Cl. CPC F04D 25/088; F24F 8/22; F04D 29/703

(57) ABSTRACT

An apparatus is for cleaning and circulating air within a room including a ceiling. A fan includes a hub connected to a plurality of blades for circulating the air, and a motor for rotating the plurality of blades about an axis. A cleaner is adapted for mounting to the ceiling for cleaning air for delivery to the fan. The cleaner includes a tube for delivering air from an intake to a discharge for being circulated by the or more of the plurality of blades. The tube includes an energy source for emitting energy sufficient to kill a living organism in the air.

This application claims the benefit of U.S. Provisional Patent Application No. 63/025,621, filed May 15, 2020, U.S. Provisional Patent Application Ser. No. 63/029,105, filed May 22, 2020, U.S. Provisional Patent Application Ser. No. 63/038,446, filed Jun. 12, 2020, U.S. Provisional Patent Application Ser. No. 63/045,882, filed Jun. 30, 2020, and U.S. Provisional Patent Application Ser. No. 63/060,826, filed Aug. 4, 2020, the disclosures of which are incorporated herein by reference.

### BACKGROUND

A variety of fan systems have been made and used over the years in a variety of contexts. For instance, various ceiling fans are disclosed in U.S. Pat. No. 7,284,960, entitled “Fan Blades,” issued Oct. 23, 2007; U.S. Pat. No. 6,244,821, entitled “Low Speed Cooling Fan,” issued Jun. 12, 2001; U.S. Pat. No. 6,939,108, entitled “Cooling Fan with Reinforced Blade,” issued Sep. 6, 2005; and U.S. Pat. No. D607,988, entitled “Ceiling Fan,” issued Jan. 12, 2010. The disclosures of each of those U.S. patents are incorporated by reference herein. Additional exemplary fans are disclosed in U.S. Pat. Pub. No. 2008/0008596, entitled “Fan Blades,” published Jan. 10, 2008; U.S. Pat. Pub. No. 2009/0208333, entitled “Ceiling Fan System with Brushless Motor,” published Aug. 20, 2009; and U.S. Pat. Pub. No. 2010/0278637, entitled “Ceiling Fan with Variable Blade Pitch and Variable Speed Control,” published Nov. 4, 2010, the disclosures of which are also incorporated by reference herein. It should be understood that teachings herein may be incorporated into any of the fans described in any of the above-referenced patents, publications, or patent applications. It should also be understood that a fan may include sensors or other features that are used to control, at least in part, operation of a fan system. For instance, such fan systems are disclosed in U.S. Pat. Pub. No. 2009/0097975, entitled “Ceiling Fan with Concentric Stationary Tube and Power-Down Features,” published Apr. 16, 2009, the disclosure of which is incorporated by reference herein; U.S. Pat. Pub. No. 2009/0162197, entitled “Automatic Control System and Method to Minimize Oscillation in Ceiling Fans,” published Jun. 25, 2009, the disclosure of which is incorporated by reference herein; U.S. Pat. Pub. No. 2010/0291858, entitled “Automatic Control System for Ceiling Fan Based on Temperature Differentials,” published Nov. 18, 2010, the disclosure of which is incorporated by reference herein; and U.S. Provisional Patent App. No. 611165,582, entitled “Fan with Impact Avoidance System Using Infrared,” filed Apr. 1, 2009, the disclosure of which is incorporated by reference herein. Alternatively, any other suitable control systems/features may be used in conjunction with embodiments described herein.

In some environments, it is desirable to clean the air in the same room as the fan. This may be achieved through the use of germicidal energy, such as UV light (which is often referred to as ultraviolet germicidal irradiation (UVGI)). UVGI is a disinfection method that uses ultraviolet (UV) light at sufficiently short wavelength to kill living microorganisms. It is used in a variety of applications, such as food, air and water purification. UVGI utilizes short-wavelength ultraviolet radiation (UV-C) that is harmful to microorganisms. It is effective in destroying the nucleic acids in these organisms so that their DNA is disrupted by the UV radiation, leaving them unable to perform vital cellular functions.

As can be appreciated, a stationary germicidal fixture positioned in a space is somewhat effective, but obviously limited in efficacy given its stationary nature (and the use of multiple stationary devices is considered costly and inefficient in most applications). In an effort to address this shortcoming, others have in the past proposed connecting UV devices to ceiling fans, but significant limitations remain. For instance, U.S. Pat. No. 4,422,824 proposes incorporating a light into a fan blade, which obviously impacts the appearance and aerodynamic features of the blade, and otherwise makes for a complicated arrangement.

Accordingly, a need is identified for an improved manner of providing a fan with a sterilizing capability and, in particular, a strategically mounted air cleaner.

### SUMMARY

According to one aspect of the disclosure, an apparatus for cleaning and circulating air within a room including a ceiling. The apparatus includes a fan including a hub connected to a plurality of blades for circulating the air. A cleaner is adapted for mounting to the ceiling for cleaning air for delivery to the fan, the cleaner comprising a tube for delivering air from an intake to a discharge for being circulated by the or more of the plurality of blades. The tube includes an energy source for emitting germicidal energy, sufficient to kill a living organism in the air.

In one embodiment, the tube is positioned above the blades and oriented coaxially with the axis. A fan may be provided adjacent the intake for causing air to move from the intake to the discharge. The tube may include a filter, such as at a discharge end.

The energy source may comprise a sterilizer, such as a UV source with at least one bulb for emitting UV-C radiation. The bulb may be elongated in an axial direction. Alternatively, an ion generator may be used to provide germicidal or sterilizing energy.

The apparatus may further include a support for supporting the ceiling fan from the tube, and a mount for mounting the tube to the ceiling. The tube may include an interior surface with a reflective coating.

According to another aspect of the disclosure, an apparatus for circulating air is provided. The apparatus comprises a ceiling fan and a downtube including an air passage with an outlet adjacent to the ceiling fan.

In one embodiment, the downtube includes a fan at an intake for causing air to flow through the air passage. A sterilizer may be positioned within the downtube. The downtube may be connected to the ceiling fan and may have a diameter corresponding to a diameter of a hub of the ceiling fan. The downtube may include a filter, and the fan may include an oversized housing connected to the downtube. The downtube may include an interior surface with a reflective coating.

Still a further aspect of the disclosure pertains to an apparatus for circulating air in a room including a ceiling. The apparatus comprises a fan and a support for the ceiling fan from the ceiling. The support includes an airflow passage for delivering air from an intake to an exhaust positioned above an upper surface of the fan.

In one embodiment, a cleaner is provided for cleaning the air in the airflow passage. The support may comprise a tube including an interior surface with a reflective coating.

### BRIEF DESCRIPTION OF THE DRAWINGS

The accompanying drawing figures incorporated herein and forming a part of the specification, illustrate several aspects of the disclosure and, together with the description, serve to explain certain principles thereof. In the drawing figures:

FIG. 1 is a side view of a fan with an air cleaner; and

FIG. 2 is a cross-sectional view of a fan with an air cleaner.

The following description of certain examples of the invention should not be used to limit the scope of the present disclosure. Other examples, features, aspects, embodiments, and advantages of the invention will become apparent to those skilled in the art from the following description, which includes by way of illustration, one or more of the best modes contemplated for carrying out the invention. As will be realized, the invention is capable of other different and obvious aspects, all without departing from the invention. Accordingly, the drawings and descriptions should be regarded as illustrative in nature and not restrictive.

### DETAILED DESCRIPTION

The following description of certain examples of the invention should not be used to limit the scope of the present invention. Other examples, features, aspects, embodiments, and advantages of the invention will become apparent to those skilled in the art from the following description, which includes by way of illustration, one or more of the best modes contemplated for carrying out the invention. As will be realized, the invention is capable of other different and obvious aspects, all without departing from the invention. Accordingly, the drawings and descriptions should be regarded as illustrative in nature and not restrictive.

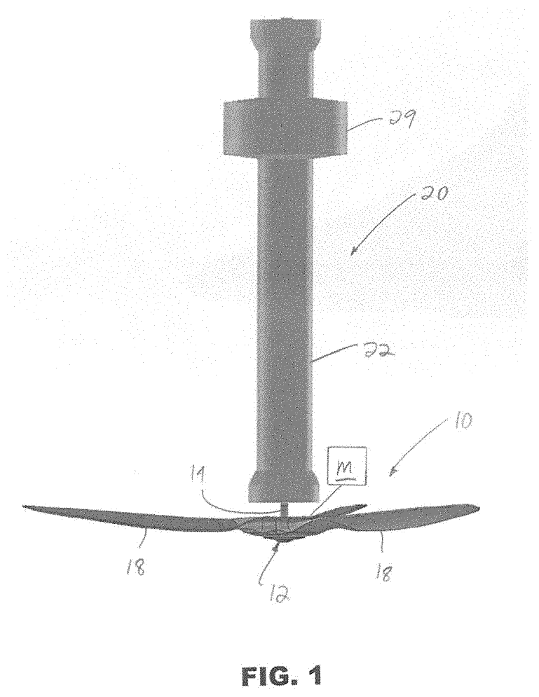

Referring to FIG. 1, a fan 10 of the present example comprises a hub 12, which may include a motor (not shown, which may be enclosed or contained within or adjacent to hub 12). The fan 10 may be connected to a support 14, and may also include a plurality of fan blades 18. In the present example, fan 10 (including hub 12 and fan blades 18) has a diameter of approximately 2-8 feet. In other variations, fan 10 has a diameter of up to 24 feet. Alternatively, fan 10 may have any other suitable dimensions depending on a particular application.

The motor M may comprise an AC induction motor having a drive shaft, though it should be understood that motor may alternatively comprise any other suitable type of motor (e.g., a permanent magnet brushless DC motor, a brushed motor, an inside-out motor, etc.). In the present example, motor M is fixedly coupled to support 14 and rotatably coupled to hub 12. Furthermore, motor is operable to rotate hub 12 and the plurality of fan blades 18. By way of example only, motor may be constructed in accordance with at least some of the teachings of U.S. Pat. Pub. No. 2009/0208333, entitled “Ceiling Fan System with Brushless Motor,” published Aug. 20, 2009, the disclosure of which is incorporated by reference herein. Furthermore, fan 10 may include control electronics that are configured in accordance with at least some of the teachings of U.S. Pat. Pub. No. 2010/0278637, entitled “Ceiling Fan with Variable Blade Pitch and Variable Speed Control,” published Nov. 4, 2010, the disclosure of which is incorporated by reference herein. Alternatively, motor may have any other suitable components, configurations, functionalities, and operability, as will be apparent to those of ordinary skill in the art in view of the teachings herein.

Hub 12 may be constructed in accordance with at least some of the teachings of U.S. Pat. Pub. No. 2010/0278637, entitled “Ceiling Fan with Variable Blade Pitch and Variable Speed Control,” published Nov. 4, 2010, the disclosure of which is incorporated by reference herein. Alternatively, hub 12 may be constructed in accordance with any of the teachings or other patent references cited herein. Still other suitable ways in which hub 12 may be constructed will be apparent to those of ordinary skill in the art in view of the teachings herein. It should also be understood that an interface component (not shown) may be provided at the interface of each fan blade 18 and hub 12. By way of example only, such an interface component may be configured in accordance with the teachings of U.S. Pat. Pub. No. 2009/0081045, entitled “Aerodynamic Interface Component for Fan Blade,” published Mar. 26, 2009, the disclosure of which is incorporated by reference herein. Of course, such an interface component may be omitted if desired.

Fan blades 18 may further be constructed in accordance with some or all of the teachings of any of the patents, patent publications, or patent applications cited herein. For example, fan blades 18 may be configured in accordance with the teachings of U.S. Pat. No. 7,284,960, entitled “Fan Blades,” issued Oct. 23, 2007; U.S. Pat. No. 6,244,821, entitled “Low Speed Cooling Fan,” issued Jun. 12, 2001; and/or U.S. Pat. No. 6,939,108, entitled “Cooling Fan with Reinforced Blade,” issued Sep. 6, 2005. The disclosures of each of those U.S. patents are incorporated by reference herein. As another merely illustrative example, fan blades 18 may be configured in accordance with the teachings of U.S. Pat. Pub. No. 2008/0008596, entitled “Fan Blades,” published Jan. 10, 2008, the disclosure of which is also incorporated by reference herein. As yet another merely illustrative example, fan blades 18 may be configured in accordance with the teachings of U.S. Pat. Pub. No. 2010/0104461, entitled “Multi-Part Modular Airfoil Section and Method of Attachment Between Parts,” published Apr. 29, 2010, the disclosure of which is incorporated by reference herein. Alternatively, any other suitable configurations for fan blades 18 may be used in conjunction with the examples described herein. In the present example, fan blades 18 are formed of aluminum through an extrusion process such that each fan blade has a substantially uniform cross section along its length. It should be understood that fan blades 18 may alternatively be formed using any suitable material, or combination of materials, by using any suitable technique, or combination of techniques, and may have any suitable cross-sectional properties or other properties as will be apparent to one of ordinary skill in the art in view of the teachings herein.

As can be appreciated, it may be desirable to utilize exemplary fan 10 disclosed above in connection with an air cleaner 20 for purposes of this disclosure. In the illustrated embodiment, the cleaner 20 supports the fan 10, such as by connecting with the support 14, and comprises a body, such as a tubular body or tube 22. The body or tube 22 defines an airflow passage P, which may have a circular cross-section, and into or through which the support 14 may extend. The tube 22 may further include a mount 24 at the upper end for mounting to a stable support structure, such as a ceiling or joist.

The tube 22 includes opening, such as an air intake 26 at one end (for example, the upper end), and opening, such as an air discharge or exhaust 28 (for example, at the lower end). This arrangement of openings or open ends thus allows for the desired air circulation through the tube 22. However, the intake and outlet ends may be reversed, as may be the direction of rotation of the fan(s) 10 or 26.

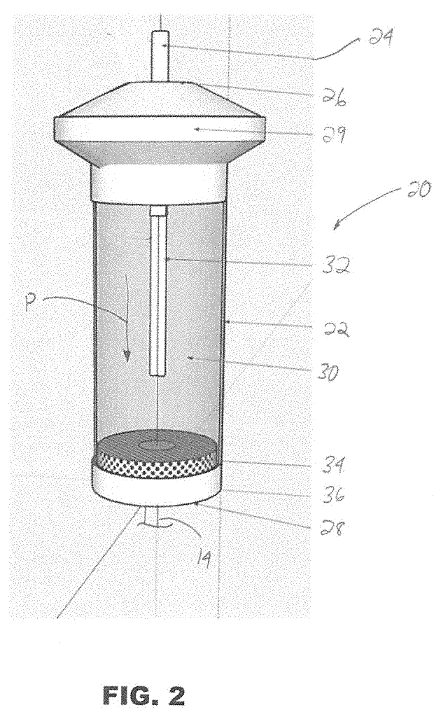

In one embodiment, as shown in FIG. 2, the tube 22 of the cleaner includes an intake fan 29 for drawing air into the intake 26, which again may be at the upper end of the cleaner 22. The intake fan 26 may be positioned in line within the tube 22, and in a separate (and slightly oversized) housing forming part of it, as shown (which may be generally in the form of a hollow cylinder, as shown in FIG. 1, or tapered, as shown in FIG. 2). The outlet or discharge opening of the tube 22 may be sized to substantially match the diameter of the hub 12, thus ensuring that the exiting air contacts the fan 10 and is distributed by the rotating fan blades 18.

While passing through the tube 22, the air encounters a source of energy for causing the demise of a living organism, such as germicidal energy. This may be provided by a sterilizer 30. The sterilizer 30 may comprise one or more UV radiation sources, such as one or more bulbs 32 supported by the mount 24 or the tube 22, but alternatively may comprise a different energy source, such as for example an ion generator connected to or positioned within the tube.

Examples of suitable UV bulbs distributed by Philips Lighting are provided in the following table:

Lamp Information

| | Length | Width | Depth | | Bulb | Current | Voltage |
|---|---|---|---|---|---|---|---|
| Model | (mm) | (mm) | (mm) | Life (h) | Shape | (A) | (V) |
| TUV-PL-S | 105 | 28 | 13 | 9000 | 2xT12 | 0.18 | 34 |
| 5W | | | | | | | |
| TUV-PL-S | 167 | 28 | 13 | 9000 | 2xT12 | 0.17 | 60 |
| 9W | | | | | | | |
| TUV-PL-S | 244 | 19 | NA | 9000 | T5 | 0.33 | 34 |
| 11W | | | | | | | |
| TUV-PL-L | 225 | 39 | 18 | 9000 | 2xT16 | 0.37 | 60 |
| 18W | | | | | | | |
| TUV-PL-L | 225 | 39 | 18 | 9000 | 2xT16 | 0.85 | 42 |
| 35W HO | | | | | | | |
| TUV-PL-L | 415 | 38 | 18 | 9000 | 2xT16 | 0.44 | 105 |
| 36W | | | | | | | |
| TUV-PL-L | 535 | 38 | 18 | 9000 | 2xT16 | 0.54 | 103 |
| 55W | | | | | | | |
| TUV-PL-L | 535 | 38 | 18 | 9000 | 2xT16 | 0.8 | 115 |
| 95W HO | | | | | | | |

The bulb(s) 32 may be elongated in a direction aligned with the axis of rotation of the fan 10, and may be powered by the same source of power for the fan motor and/or an associated light source for projecting visible light from the fan 10. The bulb(s) 32 and may be connected through electrical transmission lines extending along, within or through the support 14 and mount 24, and to a remote source (such as the AC mains). Alternatively, the bulb(s) 32 may be powered by a different source from the fan 10, such as a battery, which may be separate from the fan 10. More than one bulb 32 may be provided.

To enhance the distribution of the UV light, the interior of the body 22 may be at least partially provided with a reflective coating. Examples of reflective coatings include one or more of the following:

TABLE 1
EXAMINED REFLECTORS

| | Refl. Coeff. | Thickness | | |
|---|---|---|---|---|
| Reflector | [symbol] | [symbol] | Source | Refl. Ref. |
| SRS-99 | 0.988 | 10 | [symbol] | [symbol] * |
| (Whole Standard) | | | | [symbol] * |
| Spectralum (WS- [symbol] | 0.993 | 10 | [symbol] | [symbol] |
| Teflon ® tape (matte) | 0.99 | n × 0.06 | ACE Hardware | [18-20] |
| | | | [symbol] | |
| PTFE tape (glossy) | 0.99 | n × 0.08 | [symbol] | [18-20] |
| [symbol] ® film ( [symbol] ) | 0.99 | n × 0.16 | [symbol] | [18-20] |
| Titanium dioxide paint | 0.955 | 0.14-0.18 | [symbol] | [symbol] * |
| Magnesium oxide | 0.98 | 1.0 | [symbol] | [11-15] |
| GORE ® diffuse reflector | 0.99 | 0.50 | [symbol] | [symbol] * |
| | | | Newark, DE | |
| Numcellulose | 1.02 | 0.52 | [symbol] | [symbol] |
| Luminex ® | 0.98 | 0.24 | [symbol] , Japan | [7] |
| Metinex ® | 0.98 | 0.125 | [symbol] | [7] |
| | | | Wilmington, DE | |
| Tyvek ® paper | 0.97 | 0.11 | [symbol] | [7] |
| | | | Wilmington, DE | |
| ESR film | 0.985 | 0.065 | 3M | [symbol] *, [25] |
| | | | St. Paul, MN | |
| Aluminum foil | 0.98 | 0.025 | [symbol] | [7] |

*the reflection coefficient data provided by the manufacturer were tried [symbol]
the reflection coefficient scan measured to be 103% of the reflection coefficient of four layers of ACE Teflon ® tape at [symbol] (i.e.. 1.03 × 0.99)[7] [symbol]
indicates data missing or illegible when filed

The particular selection made depends on the particular situation, including possibly the wavelength(s) of the UV light used. One possible example involves applying Teflon tape to the interior surfaces of the tube 22.

The cleaner 20 may also include one or more filters 34. In the embodiment of FIG. 2, a single filter 34 is shown being positioned at or adjacent to the discharge 28, and thus may provide a secondary level of cleaning for the air flowing through the cleaner 20 prior to exiting and encountering the rotating blades 18 of fan 10 (including by removing organisms killed by the sterilizer 30, which may be allergens). The filter 34 may also allow for fluid or air flow, but not light, thus ensuring that the UV rays do not emanate and create a strobe effect as a result of the rotation of the fan blades 18 (or a separate cover 36 may be provided, which may allow for air flow but not light emissions). The filter 34 could also be associated with the air intake 26, or a second filter may be provided for this purpose. The filter 34 may be, for example, a HEPA filter.

The tube 22 may be made of any material, but those that are resistant to degradation as the result of prolonged exposure to radiation, such as metals, are good choices to provide a robust service life. Lightweight materials are also desirable. The tube 22 may also be provided with a removable panel (not shown) with a releasable (e.g., magnetic) latch to allow for easy access to the interior for cleaning or replacing the light, filter, etc. (which will likely have relatively short service lives and thus may need replacement.

Having shown and described various embodiments, further adaptations of the apparatuses, methods and systems described herein may be accomplished by appropriate modifications by one of ordinary skill in the art without departing from the scope of the disclosure. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the disclosure should be considered in terms of claims that may be presented, and is understood not to be limited to the details of structure and operation shown and described in the specification and drawings.

1. An apparatus for cleaning and circulating air within a room including a ceiling comprising:
a fan including a hub connected to a plurality of blades for circulating the air, and a motor for rotating the plurality of blades about an axis; and
a cleaner adapted to mounting to the ceiling for cleaning air for delivery to the fan, the cleaner comprising a tube for delivering air from an intake to a discharge for being circulated by the or more of the plurality of blades, the tube including an energy source for emitting energy sufficient to kill a living organism in the air.

2. The apparatus of claim 1, wherein the tube is positioned above the blades and oriented coaxially with the axis.

3. The apparatus of claim 1, further including another fan for causing air to move from the intake to the discharge.

4. The apparatus of claim 1, wherein the energy source comprises a sterilizer.

5. The apparatus of claim 4, wherein the sterilizer comprises a UV source with at least one bulb for emitting UV-C radiation.

6. The apparatus of claim 4, wherein the at least one bulb is elongated in an axial direction.

7. The apparatus of claim 1, wherein the tube includes a filter at a discharge end.

8. The apparatus of claim 1, further including:
a support for supporting the ceiling fan from the tube; and
a mount for mounting the tube to the ceiling.

9. The apparatus of claim 1, wherein the tube includes a reflective coating.

10. An apparatus for circulating air, comprising:
a ceiling fan; and
a downtube including an air passage with an outlet adjacent to the ceiling fan.

11. The apparatus of claim 10, wherein the downtube includes a fan for causing air to flow through the air passage.

12. The apparatus of claim 11, wherein the fan includes an oversized housing connected to the downtube.

13. The apparatus of claim 10, further including a sterilizer positioned within the downtube.

14. The apparatus of claim 10, wherein the downtube is connected to the ceiling fan.

15. The apparatus of claim 10, wherein the downtube has a diameter corresponding to a diameter of a hub of the ceiling fan.

16. The apparatus of claim 10, wherein the downtube includes a filter.

17. The apparatus of claim 10, wherein the downtube includes a reflective coating.

18. An apparatus for circulating air in a room including a ceiling, comprising:
a fan; and
a support for supporting the ceiling fan from the ceiling, the support including an airflow passage for delivering air from an intake to an exhaust positioned above the fan.

19. The apparatus of claim 17, further including a cleaner for cleaning the air in the airflow passage.

20. The apparatus of claim 18, wherein the support comprises a tube having a reflective coating.
